(19) United States
(12) Patent Application Publication
(10) Pub. No.: US 2018/0200468 A1
(43) Pub. Date: Jul. 19, 2018

(54) PATIENT INTERFACE DEVICE FABRIC SUPPORT SLEEVE

(71) Applicant: KONINKLIJKE PHILIPS N.V., EINDHOVEN (NL)

(72) Inventor: LAUREN PATRICIA CHODKOWSKI, PITTSBURGH, PA (US)

(21) Appl. No.: 15/105,590

(22) PCT Filed: Dec. 18, 2014
(86) PCT No.: PCT/IB2014/067049

§ 371 Date: Jun. 17, 2016

Related U.S. Application Data
(60) Provisional application No. 61/917,420, filed on Dec. 18, 2013.

(51) Int. Cl. A61M 16/06

(52) U.S. Cl. CPC A61M 16/0683; A61M 16/0611

(57) ABSTRACT

A support sleeve (120, 120′, 220, 220′, 320, 320′) is structured for use with a patient interface device (8) having a mask and a cushion (100, 200, 300) structured to contact a patient's face, the support sleeve including a mask covering portion (124, 224, 324) structured to surround the mask and a support strap (126, 226, 326) structured to extend across a portion of a face-contacting side of the cushion and connect opposite sides of the mask covering portion. A support sleeve (420) is structured for use with a nasal type patient interface device having a mask and a cushion structured to contact a patient's face, wherein the support sleeve comprises a mask covering portion (424) structured to surround the mask, and a front portion (426) structured to contact the bridge of the patient's nose, wherein the cushion includes an apex structured to contact the bridge of the patient's nose, and wherein the front portion extends below the apex and is structured to be disposed between the nose and the apex. A support sleeve (520) is structured for use with a cradle type patient interface having a cradle to fit beneath the nares, the sleeve comprising: a cradle covering portion (522) structured to surround the cradle; and a nose support strap (524) extending from the cradle covering portion, wherein the nose support strap is structured to extend over the nose such that the nose is disposed between the nose support strap and the cradle.

### CROSS-REFERENCE TO RELATED APPLICATIONS

This patent application claims the priority benefit under 35 U.S.C. § 119(e) of U.S. Provisional Application No. 61/917,420 filed on Dec. 18, 2013, the contents of which are herein incorporated by reference.

### BACKGROUND OF THE INVENTION

### 1. Field of the Invention

The present invention pertains to patient interface devices structured to deliver a flow of breathing gas to a patient, and, in particular, to a support sleeve for use with patient interface devices.

### 2. Description of the Related Art

Obstructive sleep apnea (OSA) is a condition that affects millions of people from around the world. OSA is characterized by disturbances or cessation in breathing during sleep. OSA episodes result from partial or complete blockage of airflow during sleep that lasts at least 10 seconds and often as long as 1 to 2 minutes. In a given night, people with moderate to severe apnea may experience complete or partial breathing disruptions as high as 200-500 per night. Because their sleep is constantly disrupted, they are deprived of the restorative sleep necessary for efficient functioning of body and mind. This sleep disorder has also been linked with hypertension, depression, stroke, cardiac arrhythmias, myocardial infarction and other cardiovascular disorders. OSA also causes excessive tiredness.

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because patient interface devices are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. Additionally, an improperly fitted mask can cause red marks or pressure sores on the face of the patient. Another concern is that an improperly fitted patient interface device can include gaps between the patient interface device and the patient that cause unwanted leakage and compromise the seal between the patient interface device and the patient. A properly fitted patient interface device should form a robust seal with the patient that does not break when the patient changes positions or when the patient interface device is subjected to external forces. Thus, it is desirable to properly fit the patient interface device to the patient.

Even when a patient interface device is properly fitted to the patient, some issues may still arise regarding the robustness of the seal and the comfort of the patient interface device. One example of such an issue is with a cradle type cushion that sits under the patient's nose. With this type of patient interface device, downward pressure from the delivery conduit can cause the seal between the patient and the cushion to be compromised. Another example occurs with full face masks or styles of nasal/oral masks. With this type of patient interface device, there is a long edge of the cushion between the bridge of the patient's nose and the edge of the patient's mouth that is unsupported. This long unsupported edge can become deformed or loose and compromise the seal between the cushion and the patient, which is sometime referred to as a blow-out. Also, full face masks have a tendency to ride up on the patient which can cause discomfort and compromise the seal. Moreover, in many types of masks where the seal between the cushion and the patient contacts the nose bridge, redness or irritation can occur on the bridge of the nose due to rubbing and undistributed forces.

Accordingly, a need exists for a mechanism for a patient interface device that improves upon existing devices to, for example, maximize patient comfort while minimizing leaks.

### SUMMARY OF THE INVENTION

In one embodiment, a support sleeve is structured for use with a patient interface device having a mask and a cushion structured to contact a patient's face. The support sleeve includes a mask covering portion structured to surround the mask and a support strap structured to extend across a portion of a face-contacting side of the cushion and connect opposite sides of the mask covering portion.

In another embodiment, a support sleeve is structured for use with a nasal type patient interface device having a mask and a cushion structured to contact a patient's face. The support sleeve includes a mask covering portion structured to surround the mask and a front portion structured to cover the cushion. The cushion includes an apex structured to contact the bridge of the patient's nose, and wherein the front portion extends below the apex and is structured to be disposed between the patient's nose and the apex.

In another embodiment, a support sleeve is structured for use with a cradle type patient interface device having a cradle structured to fit beneath a patient's nares. The support sleeve includes a cradle covering portion structured to surround the cradle and a nose support strap extending from the cradle covering portion. The the nose support strap is structured to extend over the patient's nose such that the patient's nose is disposed between the nose support strap and the cradle.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic/isometric view of a system adapted to provide a regimen of respiratory therapy to a patient according to one exemplary embodiment of the disclosed concept;

FIGS. 2A and 2B are isometric views of configurations of support sleeves for use with nasal/oral type patient interface devices according to exemplary embodiments of the disclosed concept;

FIGS. 3A and 3B are isometric views of configurations of support sleeves for use with nasal/oral type patient interface devices according to other exemplary embodiments of the disclosed concept;

FIGS. 4A and 4B are isometric views of configurations of support sleeves for use with nasal/oral type patient interface devices according to exemplary embodiments of the disclosed concept;

FIG. 5 is an isometric view of a configuration of a support sleeve for use with a nasal type patient interface device in accordance with another exemplary embodiment of the disclosed concept;

FIG. 6A is an isometric view of a cradle type patient interface device including a support sleeve in accordance with an exemplary embodiment of the disclosed concept;

FIG. 6B is an isometric view of a cradle for use with the patient interface device of FIG. 6A; and

FIG. 6C is an isometric view of the support sleeve of FIG. 6A.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

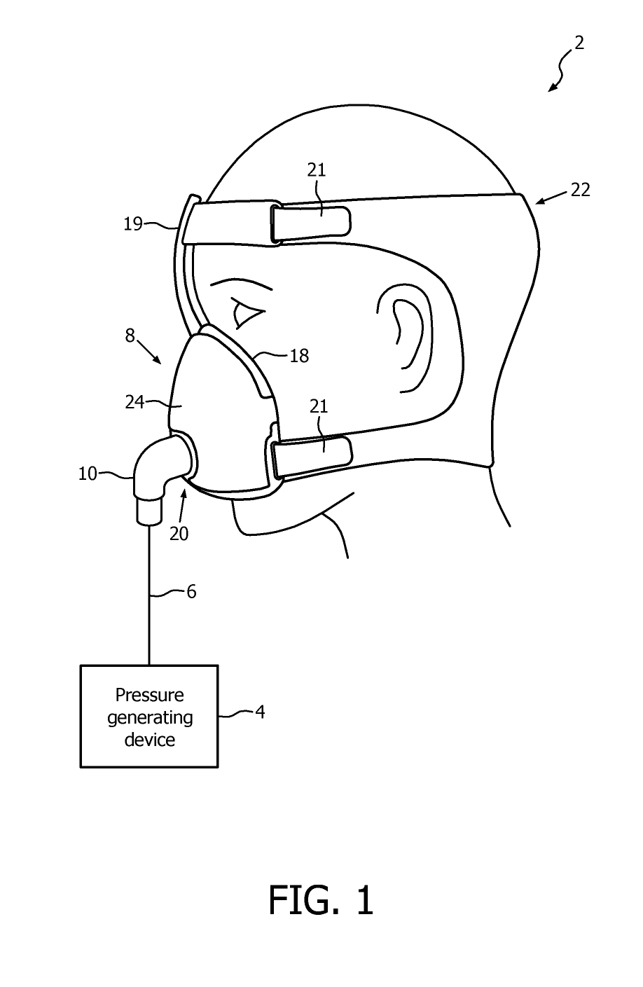

A system 2 adapted to provide a regimen of respiratory therapy to a patient according to one exemplary embodiment of the disclosed concept is generally shown in FIG. 1. System 2 includes a pressure generating device 4, a delivery conduit 6, and a patient interface device 8 including an elbow connector 10 fluidly coupled to delivery conduit 6.

Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8.

In the illustrated embodiment, patient interface 8 is a nasal/oral mask structured to cover the nose and mouth of the patient. However, any type of patient interface device 8, such as, without limitation, a nasal mask that covers the patient's nose, a nasal cushion having nasal prongs that are received within the patient's nares, or a full face mask that covers the patient's face, which facilitates the delivery of the flow of breathing gas to, and the removal of a flow of exhalation gas from, the airway of a patient may be used while remaining within the scope of the present disclosed concept. In the embodiment shown in FIG. 1, patient interface device 8 includes a rigid or semi-rigid mask 18 and a flexible cushion (hidden behind mask 18). An opening in mask 18 to which elbow connector 10 is coupled allows the flow of breathing gas from pressure generating device 4 to be communicated to an interior space defined by mask 18 and cushion, and then, to the airway of a patient. Mask 18 also includes a forehead portion 19 that is structured to contact the patient's forehead area via a forehead cushion.

Straps 20 of a headgear component 22 are attached to mask 18 to secure patient interface device 8 to the patient's head. Straps 20 may be adjusted to tighten or loosen the fit of patient interface device 8 to the patient.

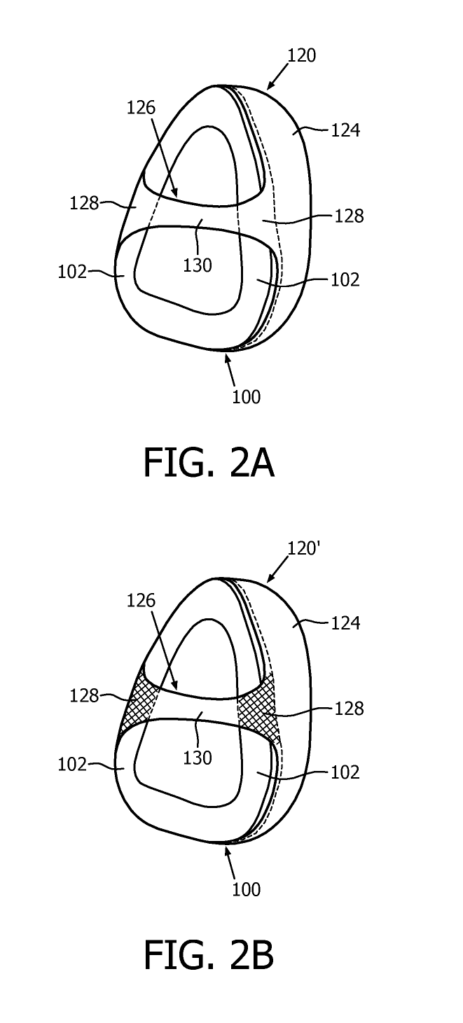

Patient interface device 8 also includes a support sleeve 24 that is structured to surround the nasal/oral portion of mask 18. Support sleeve 24 is made at least in part of fabric. Support sleeve 24 also has some elasticity which helps it conform to and fit over mask 18. Support sleeve 24 is also removable and can be taken off mask 18 and placed back on mask 18 or used with another patient interface device as desired. Support sleeve 24 shown in FIG. 1 illustrates one configuration of support sleeve 24. However, it is contemplated that support sleeve 24 may have various different configurations without departing from the scope of the disclosed concept. Furthermore, it is contemplated that different configurations of support sleeve 24 may be employed with different types of patient interface devices (e.g., nasal/oral, nasal, cradle type, etc.) without departing from the scope of the disclosed concept. Various different configurations of support sleeves will be described hereinafter with respect to FIGS. 2A-6C.

FIGS. 2A and 2B illustrate two configurations of a support sleeve 120,120′ suitable for use with a nasal/oral type patient interface device such as patient interface device 8 illustrated in FIG. 1. Referring to FIG. 2A, support sleeve 120 is fitted over a rigid or semi-rigid mask (hidden behind support sleeve 120) and a cushion 100. The face-contacting side of cushion 100 is structured to form a seal with the patient's face and includes elongated sides 102 that are structured to extend from approximately the edges of the patient's mouth to the bridge of the patient's nose.

Support sleeve 120 includes a mask covering portion 124 that is structured to surround the mask. Support sleeve 120 also includes a support strap 126 that connects opposite sides of mask covering portion 124 and extends across the face-contacting side of cushion 100 at middle portions of elongated sides 102. Support strap 126 is structured to rest under the patient's nares and in the area of the patient's upper lip when cushion 100 is placed on the patient's face. Support strap 126 provides support for elongated sides 102 of cushion 100, thus providing a more robust seal. Also, by sitting under patient's nares, support strap 126 helps to prevent cushion 100 from riding up on the patient's face.

Support strap 126 includes end portions 128 that are disposed over elongated sides 102 and a central portion 130 that extends between inner edges of elongated sides 102. In some exemplary embodiments of the disclosed concept, such as the one shown in FIG. 2B, the backside of end portions 128 of support strap 126 (i.e., the side of support strap 126 that contacts cushion 100) are coated with silicon. The backside of end portions 128 of support strap 126 may be coated with silicon using any suitable method such as, without limitation, printing the silicon on the backside of end portions 128 of support strap 126. The silicon may be a low durometer silicon (e.g., without limitation, 5-10 shA) which is elastic and slightly tacky. Coating end portions 128 with the low durometer silicon helps support sleeve 120′ stick to cushion 100 and remain in position. It is also contemplated that other portions of support sleeve 120′ may be coated with silicon such as, without limitation, the entire support strap 126 and/or mask covering portion 124 without departing from the scope of the disclosed concept.

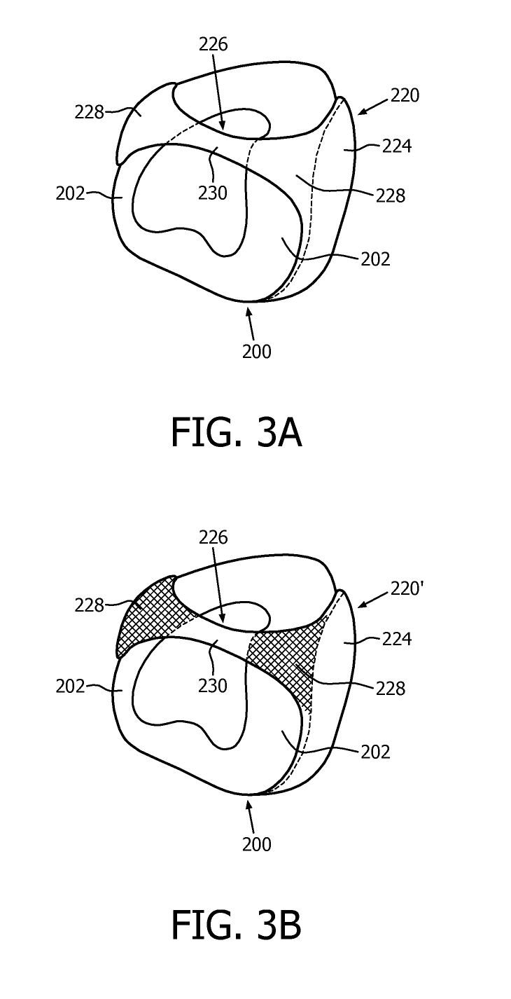

Referring to FIGS. 3A and 3B, two configurations of a support sleeve 220,220′ in accordance with exemplary embodiments of the disclosed concept are shown. Support sleeves 220,220′ are suitable for use with a nasal/oral type patient interface device similar to patient interface device 8 shown in FIG. 1. However, in FIGS. 3A and 3B, a cushion 200 and mask (hidden behind support sleeve 220,220′) are structured to rest under the patient's nares rather than extend up to the bridge of the patient's nose. Cushion 200 includes bent sides 202 which include a bend at approximately their mid portion in order to facilitate fitting cushion 200 under the patient's nares.

Support sleeves 220,220′ include a mask covering portion 224 that is structured to surround the mask. Support sleeves 220,220′ also include a support strap 226 that connects opposite sides of mask covering portion 224 and extend across the face-contacting side of cushion 200 at approximately the bend in bent sides 202. Support strap 226 is structured to rest under the patient's nares and in the area of the patient's upper lip when cushion 200 is placed on the patient's face. Support strap 226 provides support for bent sides 202 of cushion 200, thus providing a more robust seal. Also, by sitting under patient's nares, support strap 226 helps to prevent cushion 200 from riding up on the patient's face.

Support strap 226 includes end portions 228 that are disposed over bent sides 202 and a central portion 230 that extends between inner edges of bent sides 202. In some exemplary embodiments of the disclosed concept, such as the one shown in FIG. 3B, the backside of end portions 228 of support strap 226 (i.e., the side of support strap 226 that contacts cushion 200) are coated with silicon. The backside of end portions 228 of support strap 226 may be coated with silicon using any suitable method such as, without limitation, printing the silicon on the backside of end portions 228 of support strap 226. The silicon may be a low durometer silicon (e.g., without limitation, 5-10 shA) which is elastic and slightly tacky. Coating end portions 228 with the low durometer silicon helps support sleeve 220′ stick to cushion 200 and remain in position. It is also contemplated that other portions of support sleeve 220′ may be coated with silicon such as, without limitation, the entire support strap 226 and/or mask covering portion 224 without departing from the scope of the disclosed concept.

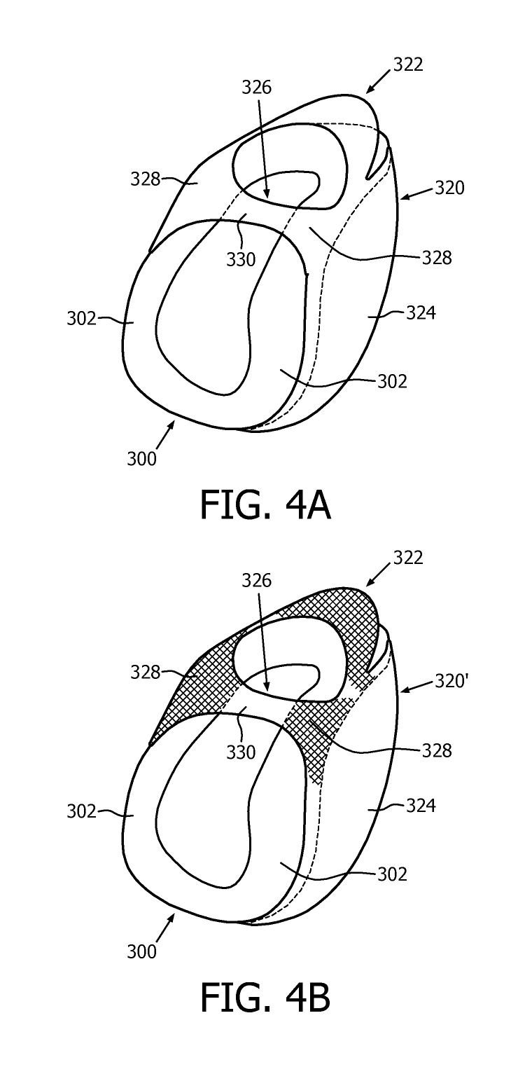

FIGS. 4A and 4B illustrate two configurations of a support sleeve 320,320′ in accordance with exemplary embodiments of the disclosed concept. Support sleeves 320,320′ are suitable to cover a cushion 300 and mask (hidden behind support sleeves 320,320′) shaped similar to cushion 200 and mask illustrated in FIGS. 2A and 2B. Cushion 300 also includes bent sides 302 which include a bend at approximately their mid portion in order to facilitate fitting cushion 300 under the patient's nares.

Support sleeves 320,320′ include a mask covering portion 324 that is structured to surround the mask. Support sleeves 320,320′ also include a support strap 326 that connects opposite sides of mask covering portion 324 and extend across the face-contacting side of cushion 300 at approximately the bend in bent sides 302. Support strap 326 is structured to rest under the patient's nares and in the area of the patient's upper lip when cushion 300 is placed on the patient's face. Support strap 326 provides support for bent sides 302 of cushion 300, thus providing a more robust seal. Also, by sitting under patient's nares, support strap 326 helps to prevent cushion 300 from riding up on the patient's face.

Support sleeves 320,320′ further include a nose support strap 322 that extends from an upper portion of support sleeves 320,320′. Nose support strap 322 is structured to fit over the patient's nose so that the patient's nose is disposed between nose support strap 322 and cushion 300 when cushion 300 and support sleeve 320,320′ are fitted to the patient. Nose support sleeve 322 prevents cushion 300 from being pulled downward such as from, for example, a downward external force from a delivery conduit.

Support strap 326 includes end portions 328 that are disposed over bent sides 302 and a central portion 330 that extends between inner edges of bent sides 302. In some exemplary embodiments of the disclosed concept, such as the one shown in FIG. 4B, the backside of end portions 328 of support strap 326 (i.e., the side of support strap 326 that contacts cushion 300) are coated with silicon. The backside of end portions 328 of support strap 326 may be coated with silicon using any suitable method such as, without limitation, printing the silicon on the backside of end portions 328 of support strap 326. The silicon may be a low durometer silicon (e.g., without limitation, 5-10 shA) which is elastic and slightly tacky. Coating end portions 328 with the low durometer silicon helps support sleeve 320′ stick to cushion 300 and remain in position.

In some exemplary embodiments such as the one shown in FIG. 4B, the backside of nose support strap 322 (i.e., the side of nose support strap 322 that contacts the patient's nose) is also coated with silicon. Coating the backside of nose support strap 322 with silicon helps nose support strap 322 to stick to the patient's nose. It is also contemplated that other portions of support sleeve 320′ may be coated with silicon such as, without limitation, the entire support strap 326 and/or mask covering portion 324 without departing from the scope of the disclosed concept.

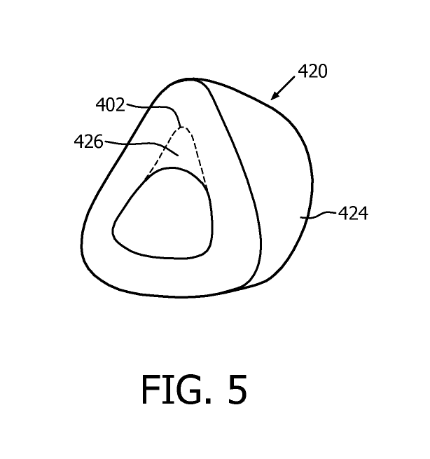

FIG. 5 illustrates a support sleeve 420 in accordance with another exemplary embodiment of the disclosed concept. Support sleeve 420 is suitable for a nasal type patient interface device. Support sleeve 420 is structured to fit over a cushion and mask (both hidden behind support sleeve 420). Support sleeve 420 includes a mask covering portion 424 that is structured to surround the mask. Support sleeve 420 also includes a front portion 426 that covers the face side of the cushion (i.e., the side of the cushion that faces the patient's face).

The cushion is structured to contact the bridge of the patient's nose at its apex 402. Contact between the cushion and the bridge of the patient's nose could cause red marks and/or irritation for the patient due to the point force applied by apex 402 to the bridge of the patient's nose. However, front portion 426 of support sleeve 420 extends lower than apex 402 which helps to distribute the force to the bridge of the patient's nose, thus increasing comfort for the patient. It will also be appreciated that a front portion that a support sleeve including a front portion that extends below an apex of a cushion to distribute the force applied to the bridge of the patient's nose can be applied to other types of patient interface devices that contact the bridge of the patient's nose without departing from the scope of the disclosed concept. For example and without limitation, it is contemplated that support sleeve 120 shown in FIG. 2A can be modified to include a front portion that extends below the apex of cushion 100 to distribute the force applied to the bridge of the patient's nose.

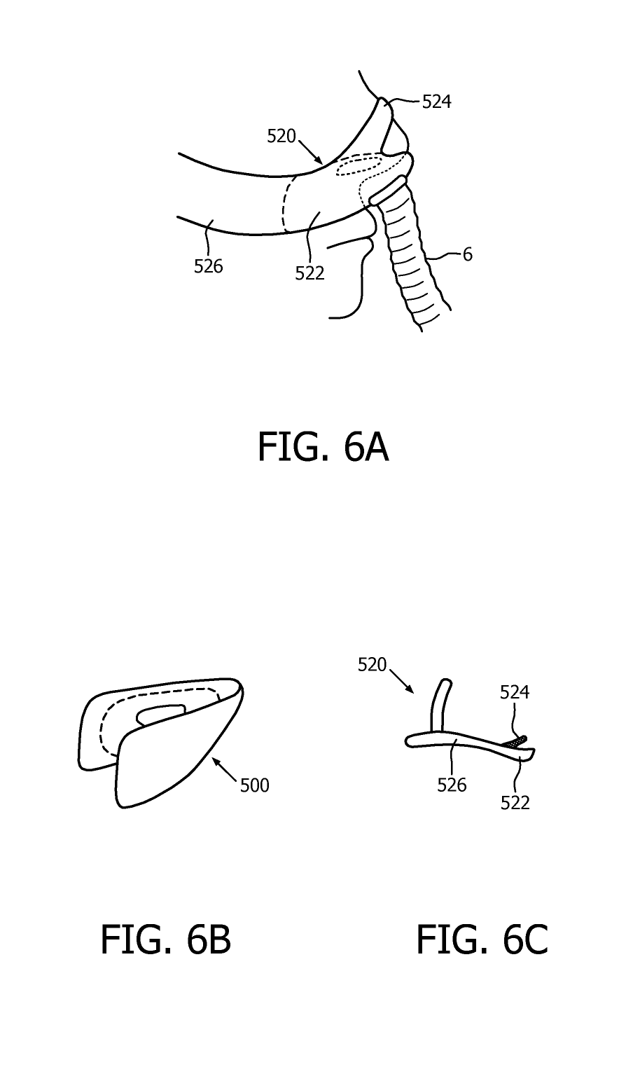

Referring to FIGS. 6A-6C a support sleeve 520 in accordance with another exemplary embodiment of the disclosed concept is illustrated. Support sleeve 520 is suitable for use with a cradle type patient interface device. Cradle type patient interface devices include a cradle that rests under the patient's nares, such as cradle 500 illustrated in FIG. 6C.

Referring to FIG. 6A, support sleeve 520 is illustrated when fitted to a patient. Support sleeve 520 is structured to surround cradle 500 (hidden behind support sleeve 520 in FIG. 6A). Support sleeve 520 includes cradle covering portion 522 that is structured to surround cradle 500. Support sleeve 520 also includes a nose support strap 524 that extends from cradle covering portion 522 and is structured to fit over the patient's nose such that the patient's nose is disposed between nose support strap 524 and cradle 500. Support sleeve 520 further includes a headgear portion 526. Headgear portion 526 extends from cradle portion 522 and is structured to extend behind and on top of the patient's head to secure support sleeve 520 to the patient (see FIG. 6C). It is also contemplated that headgear portion 526 of may be separable from cradle portion 522 without departing from the scope of the disclosed concept.

In some exemplary embodiments of the disclosed concept, the backside of nose support strap 524 (i.e., the side of nose support strap 524 that contacts the patient's nose) is coated with silicon. The backside of nose support strap 524 may be coated with silicon using any suitable method such as, without limitation, printing the silicon on the backside of nose support strap 524. Coating nose support strap 524 with silicon helps it to stick to the patient's nose. It is also contemplated that other portions of support sleeve 520 may also be coated with silicon without departing from the scope of the disclosed concept.

It is contemplated that any of the support sleeves described herein may be constructed from fabric or a blend of fabric and other materials such as, without limitation, foam. It is contemplated that any suitable fabric material may be used in the support sleeves such as, without limitation, poly-blend, cotton, or poly-spandex. It is also contemplated that the fabric material may be knit or woven. Fabric is generally more comfortable and breathable than silicon when touching the patient's face. As such, portions of support sleeves that contact the patient's face, such as support straps, are more comfortable for the patient compared to if the support straps were molded parts of the cushion.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A support sleeve for a patient interface device having a mask and a cushion, the cushion having two sides and an opening disposed between the two sides and being structured to receive the patient's nose and mouth, the support sleeve comprising:
a mask covering portion structured to surround the mask; and
a support strap structured to extend across the opening of the cushion between middle portions of the two sides of the cushion to connect opposite sides of the mask covering portion, wherein the support strap is structured to rest between the patient's face and a portion of the opening when the patient interface device is worn by the patient.

2. The support sleeve of claim 1, wherein the support strap is structured to rest against an upper lip area of the patient under the patient's nares when the patient interface device is worn by the patient.

3. The support sleeve of claim 1, wherein end portions of the support strap contact the cushion, and wherein sides of the end portions of the support strap that contact the cushion are coated with silicone.

4. The support sleeve of claim 3, wherein the silicone coated on the end portions of the support strap has a durometer in a range of about 5 shA to about 10 shA.

5. The support sleeve of claim 1, wherein the two sides of the cushion include a first elongated side and a second elongated side opposite the first elongated side, wherein each of the first and second elongated sides is structured to extend from approximately a respective corner of a patient's mouth to the bridge of the patient's nose, and wherein the support strap extends across the face-contacting side of the cushion at approximately middle portions of the first and second elongated sides.

6. The support sleeve of claim 1, wherein two sides of the cushion include a first bent side and a second bent side opposite the first bent side, wherein each of the first and second bent sides includes a bend, and wherein the support strap extends across the face-contacting side of the cushion at approximately the bends in the first and second bent sides.

7. The support sleeve of claim 1, further comprising a nose support strap extending from the mask covering portion, wherein the nose support strap is structured to extend over the patient's nose such that the patient's nose is disposed between the nose support strap and the cushion.

8. The support sleeve of claim 7, wherein a side of the nose support strap that is structured to contact the patient is coated with silicone.

9. The support sleeve of claim 8, wherein the silicone coated on the nose support strap has a durometer in a range of about 5 shA to about 10 shA.

10. The support sleeve of claim 1, wherein at least a portion of the support sleeve is made of fabric.

11. A support sleeve for a nasal type patient interface device having a mask and a cushion, the cushion having an opening structured to receive a patient's nose, the support sleeve comprising:
a mask covering portion structured to surround the mask; and
a front portion structured to cover the cushion, wherein the cushion includes an apex structured to be disposed proximate to the bridge of the patient's nose when the nasal type patient interface is worn by the patient, and wherein the front portion extends below the apex and is structured to be disposed between the patient's nose and the apex.

12. The support sleeve of claim 11, wherein at least a portion of the support sleeve is made of fabric.

13. A support sleeve for a cradle type patient interface device having a cradle structured to fit beneath a patient's nares, the support sleeve comprising:
a cradle covering portion structured to surround the cradle; and
a nose support strap extending from the cradle covering portion, wherein the nose support strap is structured to extend over the patient's nose such that the patient's nose is disposed between the nose support strap and the cradle; and wherein the nose support strap and the cradle form an opening therebetween structured such that a tip of the patient's nose can fit through the opening.

14. The support sleeve of claim 13, wherein a side of the nose support strap that contacts the patient is coated with silicone.

15. The support sleeve of claim 14, wherein the silicone coated on the nose support strap has a durometer in a range of about 5 shA to about 10 shA.

16. The support sleeve of claim 13, further comprising:
a headgear portion extending from the cradle covering portion, wherein the headgear portion is structured to extend behind the patient's head and on top of the patient's head to secure the support sleeve to the patient.

17. The support sleeve of claim 13, wherein at least a portion of the support sleeve is made of fabric.
